(19) United States
(12) Patent Application Publication
(10) Pub. No.: US 2026/0232866 A1
(43) Pub. Date: Aug. 13, 2026

(54) POLYMER EMULSION AS A LIQUID EMBOLIC SYSTEM

(71) Applicant: Covidien LP, Mansfield, MA (US)

(72) Inventors: Athanasios Touris, Costa Mesa, CA (US); Charles S. Brault, Minneapolis, MN (US)

(21) Appl. No.: 19/534,016

(22) Filed: Feb. 9, 2026

(30) Foreign Application Priority Data
Feb. 10, 2025 (GR) 20250100114

(51) Int. Cl. A61L 24/06; A61L 24/00

(52) U.S. Cl. CPC A61L 24/06; A61L 24/001; A61L 2400/06

(57) ABSTRACT

Liquid embolic compositions, systems, and methods for occluding a treatment site are provided. In some embodiments, the liquid embolic composition comprises a biocompatible polymer, and water; wherein the biocompatible polymer comprises a hydrophobic polymer and a polar head group, and wherein the liquid embolic composition is an emulsion comprising particles of the biocompatible polymer suspended in the water, and wherein the liquid embolic composition forms a solid when in contact with an ionic solution. The liquid embolic composition may further comprise an imaging agent. The liquid embolic composition disclosed herein is for use in a neurovascular or peripheral embolization procedure such as middle meningeal artery embolization, arteriovenous malformation embolization, arteriovenous fistula embolization, and tumor embolization.

### CROSS-REFERENCE TO RELATED APPLICATION(S)

The present application claims the benefit of priority to Greek Patent Application No. 20250100114, filed Feb. 10, 2025, which is incorporated by reference herein in its entirety.

### TECHNICAL FIELD

The present technology relates to biocompatible materials, in particular, to polymeric liquid embolic compositions for use in a neurovascular or peripheral embolization procedure.

### BACKGROUND

Traditional liquid embolic systems typically suffer from a variety of shortcomings. They typically comprise dimethyl sulfoxide (DMSO), which is known to cause angioneurosis as it attacks the endothelium cells. It also causes pain to the patients during injection, which makes the use of anesthesia a necessity. In the case where older patients are treated and anesthesia is not an option, patients must tolerate pain or physicians must seek out alternative, less effective therapies.

In addition, traditional liquid embolic systems are hard to inject due to their high viscosity. Even for low viscosity embolic systems, injection through a low inner diameter microcatheter requires application of relatively high pressure to the syringe to inject the system.

Use of tantalum (Ta) as the radiopaque agent can also be problematic for traditional liquid embolic systems. Ta is known to result in significant artifacts during CT and MRI imaging, making it daunting for the physicians to assess if a lesion is fully or partially occluded with embolic material especially during scans taken as a part of follow up care.

There is a need for an improved liquid embolic system that is safe, requires a low injection force, and incorporates an imaging agent that minimizes unwanted imaging artifacts.

### BRIEF DESCRIPTION OF THE DRAWINGS

FIG. 1A is an illustrated example of a biocompatible polymer comprising a positively charged initiator to form the head group and a hydrophobic polymer chain.

FIG. 1B is an illustrated example of biocompatible polymers organizing to form a hydrophobic core of hydrophobic polymer chains and a shell of positively charged initiator molecules.

FIG. 1C is an illustrated example of suspended biocompatible polymers that organize to form particles in water.

FIG. 1D is an illustrated example of suspended particles in deionized water.

FIG. 1E is an illustrated example of the emulsion after it is added to a liquid that contains ions and shows the particles surrounded by and linked together by the ions resulting in a solid precipitate.

FIG. 2 is a photo of a beaker containing the milky white biocompatible polymer emulsion in water and is an example of the polymer emulsion.

FIG. 3A is a photo of an exemplary biocompatible polymer emulsion composition being pipetted into a beaker containing a saline solution and forming a solid precipitate.

FIG. 3B is a photo of a metal spatula probing the solid precipitate in saline solution formed after the biocompatible polymer emulsion composition was pipetted into the saline solution.

FIG. 4A is a partially schematic view of a treatment system configured in accordance with embodiments of the present technology.

FIG. 4B is an enlarged cross-sectional view of a distal portion of the treatment system of FIG. 4A with an optional occlusion device.

### DETAILED DESCRIPTION

The present disclosure relates to liquid embolic compositions configured for delivery to a treatment site in a patient's body, e.g., for use in a neurovascular or peripheral embolization procedure. In one aspect, the disclosure provides a liquid embolic composition for occluding a treatment site comprising: a biocompatible polymer, and water; wherein the biocompatible polymer comprises a hydrophobic polymer and a polar head group, and wherein the liquid embolic composition is an emulsion comprising particles of the biocompatible polymer suspended in the water, and wherein the liquid embolic composition forms a solid when in contact with an ionic solution (e.g., physiological fluids.)

The disclosed liquid embolic compositions provide many advantages over conventional liquid embolization compositions. For example, water is used as the carrier instead of DMSO or any other organic solvent. Compared to conventional compositions, the disclosed liquid embolic composition has reduced toxicity and minimizes pain to the patient during injection of the embolic material. Since use of an anesthetic is not necessary, a wider range of patients including older adults may be treated.

Instead of being dissolved, the biocompatible polymer is suspended in deionized water to form an emulsion. As the emulsion comes in contact with an ionic solution (e.g., a physiological fluid), the particles of the emulsion aggregate and eventually precipitate to form a continuous mass. Because the biocompatible polymer forms aggregates (e.g., particulates) suspended in solution rather than dissolving, there are fewer polymer chain entanglements, and thus the viscosity of the polymer suspension is low and approaches that of water (~1 cP). The low viscosity results in a low injection force needed to deliver the liquid embolic composition, which improves ease of use and increases safety margins (e.g., no accidental over-injection due to an initial strong push). In some embodiments, exposure of the emulsion to an ionic solution results in a continuous solid. In some embodiments, a continuous injection of the emulsion into an ionic solution results in a continuous solid. In some embodiments, the emulsion is injected wherein the emulsion is continuous, resulting in a continuous solid.

The solid cast is cohesive, wherein the particles hold together through attractive to intermolecular forces. Cohesion contributes to the internal strength of the cast, allowing it to maintain its form under stress or pressure.

The liquid embolic composition may further comprise an imaging agent. Instead of tantalum, a non-ionic contrast agent (e.g., iodine-based agent) is mixed with the emulsion to provide radiopacity. The contrast agent dissipates out after injection and radiopacity diminishes over time to allow for clear imaging of the treatment site during follow up sessions and reduced imaging artifacts.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The headings provided herein are for convenience only and do not interpret the scope or meaning of the claimed present technology. Embodiments under any one heading may be used in conjunction with embodiments under any other heading.

### I. LIQUID EMBOLIC COMPOSITIONS

The present technology provides an injectable liquid embolic composition that forms a soft solid suitable for partially or fully occluding a treatment site. The treatment site is one that would benefit from embolization, such as a middle meningeal artery to treat chronic subdural hematoma, an arteriovenous malformation, an arteriovenous fistula, or a tumor. Other treatment sites include liver, kidney, chest, bone, uterus, prostate, brain, thyroid, fibroids, hemorrhoids, gastrointestinal tract, and pancreas. The injectable liquid embolic composition may be used in an endovascular hemostasis procedure as well as in neurovascular and peripheral embolization procedures.

The liquid embolic composition is suitable for injection via microcatheter and has the ability to conform to the anatomy of the treatment site, solidifying to an elastic, soft solid to provide long-term occlusion. Advantages of the liquid embolic system disclosed herein include compositions that are free of organic solvents (e.g., DMSO), are aqueous-based, and easy to inject. The aqueous-based compositions eliminate the possibility of an organic solvent (e.g., DMSO) leaching into the body and the associated adverse effects. The initial low viscosity of the composition disclosed herein provides good distal penetration of the composition, e.g., via injection through a microcatheter. Another advantage of the compositions described herein is that the liquid embolic composition is a one-part system and does not have the drawbacks associated with two-part systems. A two-part system may limit the window of time to place the composition at the treatment site. For instance, after mixing the two-part system, if the mixture solidifies too quickly, the catheter may become clogged before a sufficient amount of the mixture is deployed. On the other hand, if the mixture takes too long to solidify or gel, then the delivered mixture may migrate or leak out of the treatment site resulting in unintended embolization of other sites. This could lead to unintended consequences such as stroke. The liquid embolic compositions described herein do not require mixing of two components to initiate solidification of the composition. The present compositions solidify not by mixing a two-part system, but by exposure to a sufficiently ionic solution such as physiological fluids. As discussed in the Methods section, the operator of the microcatheter can take the necessary time to place the microcatheter and deliver the composition.

The components of the disclosed compositions are selected to provide properties such as precipitating to form a solid cast only after exiting the microcatheter and contacting an ionic solution such as a physiological fluid. The term “cast” refers to the embolic composition in the context of occupying a vessel or other body lumen to be occluded. The present disclosure also provides for embolic compositions having additional agents such as fillers, contrast agents, and crosslinkers.

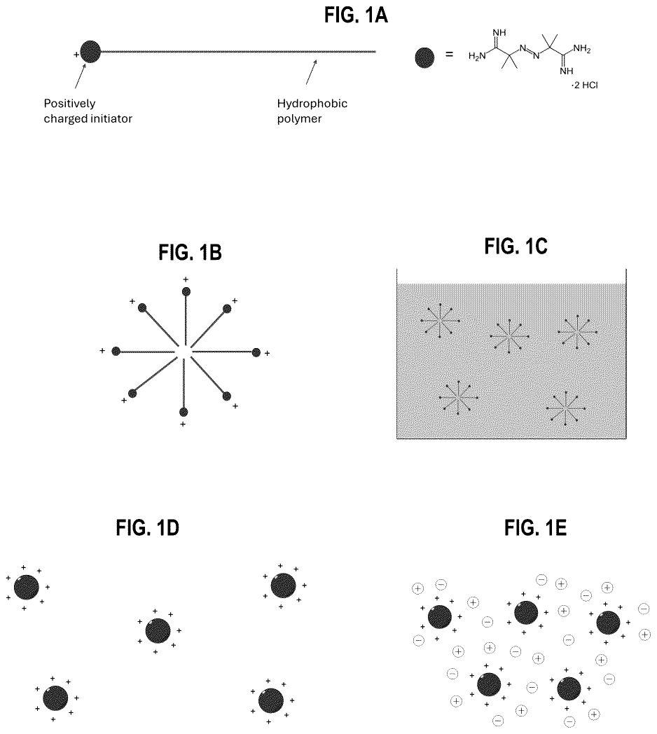

The biocompatible polymer may be described as having a head group covalently attached to a tail. The head group is attributed to the initiator selected for the polymerization reaction. The head group is polar and can be charged or uncharged. In some embodiments, the biocompatible polymer has a positively charged head group. The head group may have one or multiple charges. The tail of the biocompatible polymer has an overall hydrophobic character, which is determined by the choice of monomer selected for the polymerization reaction. FIG. 1A illustrates a non-limiting example of a biocompatible polymer comprising a positively charged head group covalently bonded to a hydrophobic polymer tail (chain). In some embodiments, the biocompatible polymer is an amphiphile, having a hydrophilic (head group) and hydrophobic (tail) portions. In some embodiments, the head group is polar and uncharged. In some embodiments the head group is polar and charged.

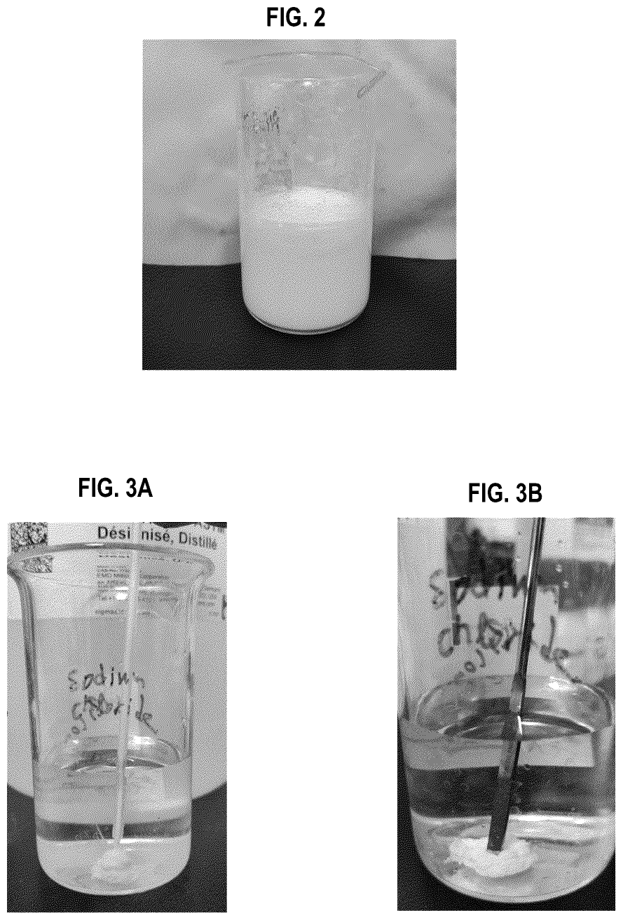

The amphiphilic nature of the biocompatible polymer permits self-assembly and formation of particulate structures. The particulate structures are composed of many individual biocompatible polymers. These particulate structures, which may also be described as aggregates, remain suspended in solution and contribute to a milky appearance. The particulate structures remain suspended because they have a charged exterior (via the positively or negatively charged head groups) and thus repel other particulate structures that have a similar charged exterior (e.g., positive repels positive, negative repels negative). As a result, the liquid embolic composition is an emulsion, comprising suspended particulate structures of the biocompatible polymer. Instead of the biocompatible polymer dissolving into the aqueous solution, the biocompatible polymer molecules organize to form particulate structures suspended in solution. The “aqueous solution” refers to a non-ionic solution or a solution having an ionic strength below physiological strength (weakly ionic.) The particulate structure consists of a hydrophobic core composed of the hydrophobic polymer chains and an outer shell composed of the polar or charged head group. This structure reduces interactions between the hydrophobic polymer chains with the aqueous environment and increases the interaction between the polar or charged head group with the aqueous environment. FIG. 1B is a 2-dimensional illustration of a number of biocompatible polymers organizing to form a core of hydrophobic chains. The charged head groups are arranged on the outside of the particle where they interact with water and form a hydrophilic shell. In other embodiments where the head group is a polar group, the biocompatible polymer may behave similarly and organize to form a hydrophobic core and a hydrophilic shell. FIG. 1C illustrates an aqueous solution containing a suspension of a number of these particulate structures, which is referred to herein as “particles.” FIG. 1D is another depiction of particles suspended in deionized water. The mutual repulsion of the positive charges on the surface of the particle promotes suspension of the particles and reduces their ability to aggregate into a clump. FIG. 2 is a photo of a beaker containing a milky white biocompatible polymer suspension in deionized water and is an example of the liquid embolic composition. The terms “suspension” and “emulsion” may be used interchangeably herein.

Once the emulsion is exposed to an ionic solution, it precipitates and forms a cohesive solid. The charged head groups of the particulates are neutralized by the ions from the ionic solution so that the repulsive force between the previously charged particles (positive charges repel) is eliminated. Without the repulsive force between the particles, the hydrophobic portions of the polymers contribute to the formation of polymer aggregates, resulting in precipitation of the biocompatible polymer. FIG. 1E illustrates the particles after it is injected into an ionic solution (e.g., blood). The particles are surrounded by ions from the liquid, linking together the positively charged microparticles, which results in aggregation and precipitation of a solid.

### A. Preparation of the Liquid Embolic Composition

The liquid embolic composition disclosed herein comprises a biocompatible polymer and water, wherein the biocompatible polymer is a polymerization product of a mixture comprising a polar radical initiator and hydrophobic monomers selected from the group consisting of acrylate, vinyl acetate, styrene, acrylonitrile, and vinyl chloride, each of which is optionally substituted.

### 1. Radical Initiator

Both the head group and the hydrophobic polymer contribute to the amphiphilic nature of the biocompatible polymer. The head group provides the polar, hydrophilic portion and the hydrophobic polymer (tail) provides the hydrophobic portion of the biocompatible polymer. Whether the polar head group is charged or uncharged, its contrasting character to the hydrophobic polymer should promote the formation of a stable emulsion in the liquid embolic system as well as the precipitation of a solid after contact with an ionic solution. The polar nature of the head group refers to an uneven distribution of electrons, which can result in a charged or uncharged molecule.

The head group of the biocompatible polymer is covalently bonded to the hydrophobic polymer portion (the hydrophobic tail) and is derived from a radical initiator. Under certain conditions such as heat or light, radical initiators generate free radicals that can trigger a chain reaction by reacting with a monomer molecule. The radical initiator is a source of free radicals that start the polymerization process and also provides the polar head group portion of the biocompatible polymer, depending on the choice of the radical initiator. In some embodiments, the radical initiator is charged. In some embodiments, the radical initiator is not charged but provides a polar head group for the polymer.

Examples of radical initiators include and are not limited to the following:
2,2′-azobis(2-methylpropionamidine)dihydrochloride,
2,2′-azobis[2-methyl-n-(2-hydroxyethyl)propionamide],
4,4′-azobis(4-cyanovaleric acid),
2,2′-azobis[n-(2-carboxyethyl)-2-methylpropionamidine]tetrahydrate,
2,2′-azobis[2-(2-imidazolin-2-yl)propane],
2,2′-azobis[2-(2-imidazolin-2-yl)propane]dihydrochloride,
potassium persulfate,
ammonium persulfate, and
sodium persulfate.

In some embodiments, the radical initiator is a salt of any one of the compounds:
2,2′-azobis(2-methylpropionamidine),
2,2′-azobis[2-methyl-n-(2-hydroxyethyl)propionamide],
4,4′-azobis(4-cyanovaleric acid),
2,2′-azobis[n-(2-carboxyethyl)-2-methylpropionamidine]tetrahydrate,
2,2′-azobis[2-(2-imidazolin-2-yl)propane], and
2,2′-azobis[2-(2-imidazolin-2-yl)propane].

In some embodiments, the radical initiator is a hydrochloride salt of any one of the compounds:
2,2′-azobis(2-methylpropionamidine),
2,2′-azobis[2-methyl-n-(2-hydroxyethyl)propionamide],
4,4′-azobis(4-cyanovaleric acid),
2,2′-azobis[n-(2-carboxyethyl)-2-methylpropionamidine]tetrahydrate,
2,2′-azobis[2-(2-imidazolin-2-yl)propane], and
2,2′-azobis[2-(2-imidazolin-2-yl)propane].

In some embodiments, the radical initiator is 2,2′-azobis(2-methylpropionamidine) dihydrochloride, or 2,2′-azobis[2-(2-imidazolin-2-yl)propane]dihydrochloride.

In some embodiments, the radical initiator may be a charged molecule, for instance 2,2′-azobis(2-methylpropionamidine)dihydrochloride, 2,2′-azobis[2-(2-imidazolin-2-yl)propane]dihydrochloride, potassium persulfate, ammonium persulfate, and sodium persulfate. The radical initiator may be a positively charged molecule or a negatively charged molecule.

In some embodiments, the radical initiator is not charged but provides a polar head group, for instance 2′-azobis[2-methyl-n-(2-hydroxyethyl)propionamide], 4,4′-azobis(4-cyanovaleric acid), 2,2′-azobis[n-(2-carboxyethyl)-2-methylpropionamidine]tetrahydrate, and 2,2′-azobis[2-(2-imidazolin-2-yl)propane.

In some embodiments, the polymerization reaction is a controlled radical polymerization and the radical initiator initiates a living polymerization reaction. In some embodiments, the polymerization reaction is a living free radical polymerization reaction such as atom transfer radical polymerization (ATRP) and reversible addition-fragmentation chain transfer (RAFT.) Examples of RAFT radical initiators are azo initiators (e.g., azobisisobutyronitrile (AIBN), 4,4′-azobis(4-cyanovaleric acid) (ACVA), 2,2′-azobis-(2,4-dimethylvaleronitrile) (ABVN)), peroxide initiators (e.g., benzoyl peroxide (BPO), lauroyl peroxide). A RAFT polymerization mixture further comprises a RAFT chain transfer agent. Non-liming examples of ATRP initiators include alkyl halides, tosyl chloride, and dimethyl 2,6-dibromoheptanedioate (DMDBHD). The RAFT and ATRP initiator may be functionalized to incorporate charged groups to impart ionic properties to the polymer, or otherwise be modified to incorporate polarity and subsequent polar head group properties to the polymer.

### 2. Initiator to Monomer Molar Ratio

The initiator to monomer ratio is the molar ratio between the radical initiator and the hydrophobic monomer in the mixture prior to polymerization and may be referred to as “initiator: monomer.” The initiator to monomer ratio impacts a variety of features of the polymerization product, in turn the liquid embolic composition and its ability to be used successfully in occluding a treatment site. Besides determining whether or not a polymerization reaction proceeds, the initiator to monomer ratio impacts the length of the polymer chains, the size of the aggregated polymer particles, the stability of the emulsion, the behavior of the emulsion when contacted with an ionic solution, and the cohesion of the solid cast. The stability of the emulsion and the ability of the emulsion to form a cohesive solid upon exposure to a physiological fluid can be tuned by modifying the initiator to monomer molar ratio.

For example, a high initiator: monomer ratio results in increased number of lower molecular weight chains (shorter chains) and possibly smaller particle sizes (polymer aggregates). In some cases, smaller particles may reduce viscosity and promote formation of a stable emulsion.

On the other hand, if the initiator: monomer ratio is too low, polymerization may not take place. Alternatively, if polymerization does occur, a low initiator: monomer ratio may produce long polymer chains that can promote precipitation upon exposure of the polymer to a physiological solution.

In some embodiments, the initiator: monomer molar ratio is about 1:3000. In some embodiments, the initiator: monomer molar ratio is 1:100 to 1:30000. In some embodiments, the initiator: monomer molar ratio is 1:1000 to 1:15000. In some embodiments, the initiator: monomer molar ratio is 1:1000 to 1:10000. In some embodiments, the initiator: monomer molar ratio is 1:1000 to 1:5000. In some embodiments, the initiator: monomer molar ratio is 1:2000 to 1:15000. In some embodiments, the initiator: monomer molar ratio is 1:2000 to 1:10000. In some embodiments, the initiator: monomer molar ratio is 1:2000 to 1:5000. In some embodiments, the initiator: monomer ratio is selected from the group consisting of 1:100 to 1:30000, 1:1000 to 1:20000, 1:1000 to 1:10000, 1:1000 to 1:5000, 1:2000 to 1:20000, 1:2000 to 1:10000, 1:2000 to 1:5000, 1:3000 to 1:20000, 1:3000 to 1:10000, 1:3000 to 1:5000, and 1:2000 to 1:4000.

In some embodiments the initiator: monomer molar ratio is 1:2000 to 1:4000, wherein the initiator is a charged radical initiator and the monomer is vinyl acetate. In some embodiments the initiator: monomer molar ratio is 1:2000 to 1:4000, wherein the initiator is 2,2′-azobis(2-methylpropionamidine)dihydrochloride or 2,2′-azobis[2-(2-imidazolin-2-yl)propane]dihydrochloride, and the monomer is vinyl acetate.

### 3. Hydrophobic Monomers

The hydrophobic monomer is selected to react with a radical initiator as disclosed herein. The resulting polymerized product has hydrophobic “tail” comprising the repeating monomer units. In some embodiments the hydrophobic monomer is an acrylate, vinyl acetate, styrenic, acrylonitrile, or vinyl chloride.

Examples of acrylate monomers include and are not limited to methyl (meth)acrylate, butyl (meth)acrylate, hexyl (meth)acrylate, octyl (meth)acrylate, 2-ethylhexyl (meth)acrylate, benzyl (meth)acrylate, cyclohexyl (meth)acrylate, hexadecyl (meth)acrylate, octadecyl acrylate, and phenyl (meth)acrylate.

Examples of styrenic monomers include and are not limited to styrene, dimethylstyrene, halogenated styrene (e.g., chlorostyrene, dichlorostyrene, 4-bromostyrene), 4-hydroxystyrene, 4-acetoxystyrene, methyl styrene, butyl styrene, ethoxystyrene, and 4-fluorostyrene.

The hydrophobic monomer concentration should promote the formation of suspended particles and a stable liquid emulsion in the polymerization product. A relatively high monomer concentration may result in a polymerization product that forms an unstable emulsion, where the suspended polymer particles form precipitates and “crashes out” of solution.

In some embodiments, the prepolymerization concentration of hydrophobic monomer in water is about 10% v/v. In some embodiments, the prepolymerization concentration of monomer in water is 7% to 12% v/v. In some embodiments, the prepolymerization concentration of monomer in water is 5% to 15% v/v. In some embodiments, the prepolymerization concentration of monomer in water is 1% to 20% v/v.

The water used in the polymerization reaction and in the liquid embolic composition promotes the formation of a stable emulsion and reduces the precipitation of the biocompatible polymer when not in contact with an ionic solution (e.g., physiological fluid.) The water has a concentration of ions that is lower than that of the ionic solution and is typically deionized water. In some embodiments, the water is at least 80% deionized. In some embodiments, the water is at least 90% deionized. In some embodiments, the water is at least 95% deionized. In some embodiments, the water is at least 99% deionized.

In some embodiments, in addition to the initiator and monomers, the reaction mixture further comprises crosslinking agents. A nonlimiting example of a crosslinking agent is a difunctional crosslinking group like a difunctional acrylate. A crosslinking agent may be included in a low concentration to improve cohesion of the final cast.

### 4. Hydrophobic Polymer

The polymerization product as discussed above is a biocompatible polymer comprising a hydrophobic polymer and a polar head group. The hydrophobic polymer is biocompatible. In some embodiments, the hydrophobic polymer is biodegradable. In some embodiments, the hydrophobic polymer is resorbable. In some embodiments, the hydrophobic polymer is resistant to degradation and is intended to be a permanent implant. In some embodiments, the hydrophobic polymer is selected from the group consisting of polyacrylate, polyvinyl acetate, polystyrene, polyacrylonitrile, and polyvinyl chloride, each of which is optionally substituted.

In some embodiments, the hydrophobic polymer is a polyacrylate selected from the group consisting of poly(methyl methacrylate, poly(butyl methacrylate), poly(hexyl methacrylate), poly(octyl methacrylate), poly(2-ethylhexyl methacrylate), poly(benzyl methacrylate), poly(cyclohexyl methacrylate), poly(hexadecyl methacrylate), poly(octadecyl acrylate), and poly(phenyl methacrylate), each of which is optionally substituted.

In some embodiments, the hydrophobic polymer is a polystyrene. In some embodiments the hydrophobic polymer is selected from the group consisting of poly(styrene), poly(dimethylstyrene), halogenated poly(styrene) (e.g., chlorostyrene, dichlorostyrene, 4-bromostyrene), poly(4-hydroxystyrene), poly(4-acetoxystyrene), poly(methyl styrene), poly(butyl styrene), poly(ethoxystyrene), and poly(4-fluorostyrene). In some embodiments, the polystyrene is substituted with one or more substituents selected from the group consisting of methyl, halogen, hydroxy, acetoxy, methyl, butyl, ethoxy, and fluoro.

### 5. Viscosity of the Liquid Embolic Composition

The viscosity of the liquid embolic composition is low enough to facilitate injection through a delivery catheter. If the viscosity is too high, great force is required to push the liquid embolic composition out of the delivery device, resulting in reduced control of the delivery of the material, increased risk of injury to the subject, and an increased risk of delivering more than the desired amount of the composition. The liquid embolic composition described herein possesses a viscosity that permits smooth, controlled, and easy (low pressure) injection.

Viscosity may be influenced by particle concentration, with higher particle concentration resulting in higher viscosity, and lower particle concentration resulting in lower viscosity. For instance, viscosity may be increased by centrifuging and condensing the emulsion. On the other hand, increasing the volume and decreasing the particle concentration reduces the viscosity.

Viscosity may also be influenced by particle size, with larger particle sizes resulting in higher viscosity, and smaller particle sizes resulting in lower viscosity. In some embodiments, the particle size of the liquid embolic composition (e.g., mean particle diameter) is 100 nm to 2000 nm. In some embodiments, the particle size of the liquid embolic composition (e.g., mean particle diameter) is 100 nm to 1000 nm. In some embodiments, the particle size of the liquid embolic composition (e.g., mean particle diameter) is 1000 nm to 2000 nm.

In some embodiments, the viscosity of the liquid embolic composition at 25° C. is 1.0 cP to 10 cP. In some embodiments, the viscosity of the liquid embolic composition at 25° C. is 1.0 cP to 5 cP. In some embodiments, the viscosity of the liquid embolic composition at 25° C. is 1.0 cP to 3 cP. In some embodiments, the viscosity of the liquid embolic composition at 25° C. is 1.0 cP to 2 cP. In some embodiments, the viscosity of the liquid embolic composition at 25° C. is 1.5 cP to 5 cP. In some embodiments, the viscosity of the liquid embolic composition at 25° C. is 1.5 cP to 3 cP. In some embodiments, the viscosity of the liquid embolic composition at 25° C. is 1.5 cP to 2 cP.

### 6. Non-Ionic Contrast Agents

In some embodiments, the liquid embolic composition further comprises an imaging agent, which helps visualize the presence and shape of the composition as well as the solid cast using radiographic techniques. In some embodiments the imaging agent is a non-ionic contrast agent. In some embodiments the non-ionic contrast agent contains iodine. Non-limiting examples of iodinated non-ionic contrast agents include iopamidol (Isovue® 300, 370 and Iopamiro® 370), iohexol (Omnipaque® 240, 300, 350), iopromide (Ultravist® 370), ioversol (Optiray® 300, 320 and 350), ioxilan (Oxilan® 350), iodixanol (Visipaque® 320), and lipiodol (Guerbet®).

Optionally, after the liquid embolic composition is implanted into the treatment site, the contrast agent may gradually diffuse out of the solid, thus resulting in diminishing radiopacity over time. This approach may be advantageous where the contrast agent produces imaging artifacts that may interfere with subsequent imaging of the treatment site. In some embodiments, the cast is configured to release at least 10%, 20%, 30%, 40%, 50%, 60%, 70%, 80%, 90%, 95%, or 99% of the initial loading contrast agent within a target time period after implantation, where the target time period is at least 1 day, 2 days, 3 days, 4 days, 5 days, 6 days, 7 days, 2 weeks, 3 weeks, 4 weeks, 1 month, 2 months, or 3 months after implantation. In other embodiments, however, the contrast agent may remain within the cast without leaching out after implantation.

In other embodiments, the imaging agent is optional and may be omitted from the liquid embolic composition. In such embodiments, the degree of occlusion of the treatment site by the cast may be assessed using other techniques.

### B. Methods of Using the Liquid Embolic Compositions

Once the liquid embolic composition is exposed or comes in contact with a precipitation medium, e.g., a liquid of sufficient ionic strength, such as a physiological fluid or 0.9% saline, the emulsion undergoes a transformation. The ions in the precipitation medium link together the positively charged microparticles causing aggregation and eventually precipitation. The ionic environment causes the suspended biocompatible polymer particles in the emulsion to aggregate with each other, eventually precipitating into a solid mass.

The precipitation medium is an ionic solution, which is a liquid mixture comprising ions dissolved in a solvent, typically water. For example, saline is an ionic solution comprising dissolved sodium chloride (NaCl) in water, which dissociates into positively charged sodium and negatively charged chlorine ions. Human blood plasma is approximately 0.9% NaCl and has an ionic strength of 0.154 mol/L. Isotonic saline has an ionic strength of 0.154 mol/L, and 154 mEq/L of sodium and 154 mEq/L of chloride. In some embodiments, the ionic solution has an ionic strength of human physiological fluid, like human blood plasma. In some embodiments the liquid embolic composition precipitates in an ionic fluid that has an ionic strength within plus or minus 5%, 10%, 15%, 20%, 25%, 30%, 35%, 40%, 45%, or 50% of 0.154 m/L. In some embodiments, the ionic solution is a physiological solution. In some embodiments, the ionic solution is blood. In some embodiments, the ionic solution is saline (e.g., 0.9% NaCl). In some embodiments, the saline solution is phosphate-buffered saline (PBS).

Until the liquid embolic composition is exposed to such ionic solution, the composition is a low-viscosity, stable emulsion that can be easily injected through a microcatheter and delivered to a treatment site in the body. The operator of the microcatheter can take the necessary time without rush to place the delivery instrument at the precise location of the treatment site before injecting the liquid embolic composition. However, once the delivery instrument is in the proper place and the liquid embolic composition is injected, contact with the physiological fluid of the body and the high ionic content triggers the transformation of the liquid embolic composition from emulsion to a solid, occlusive cast. The solid cast is cohesive and stable.

The liquid embolic composition can be used to treat conditions that would benefit from occluding a treatment site. In some embodiments, the present disclosure provides for a method for occluding a treatment site comprising delivering the liquid embolic composition to the treatment site. A treatment site is at or near an area of dysfunction, for instance, arteriovenous malformations (e.g., brain arteriovenous malformations), arteriovenous fistulas, tumors (e.g., via occlusion of vessel(s) feeding a tumor), chronic subdural hematomas (e.g., via occlusion of the middle meningeal artery).

The liquid embolic compositions described herein may be used in neurovascular or peripheral embolization procedures is selected from the group consisting of middle meningeal artery embolization, arteriovenous malformation (AVM) embolization, arteriovenous fistula (AVF) embolization, and tumor embolization. AVF is an abnormal, direct, and simple connection between an artery and a vein. On the other hand, a typical AVM has multiple connections that are complex, net-like structures.

The liquid embolic compositions described herein can be used to treat conditions related to chronic subdural hematomas (cSDH), brain or spinal arteriovenous malformations (AVMs), dural arteriovenous fistulas, pulmonary arteriovenous fistulas, cancerous tumors, and noncancerous tumors. Tumors may occur in various parts of the body including the liver, kidney, chest, bone, uterus, prostate, brain, and thyroid.

In some embodiments, formation of a solid cast at the treatment site occludes a vessel and provides beneficial effects to the patient. For example, the embolic composition can be delivered into a treatment site to partially or fully occlude the treatment site. The embolic composition can be delivered via injection, e.g., via a catheter or other elongate shaft that is introduced to the treatment site via the vasculature. Delivery treatment systems are described in Section II.

When the liquid embolic composition is exposed or is in contact with an ionic solution the time it takes to transform from a low viscosity emulsion to a solid, occlusive cast is relatively short. In some embodiments, this “solidification time” is instantaneous or within seconds. In some embodiments, the cure time is within 5 seconds. In some embodiments, the cure time is 10, 15, 20, or 30 seconds.

In some embodiments, solidification occurs without requiring a covalent crosslinking reaction or further chemical reaction. In some embodiments, solidification occurs without requiring a thermally triggered phase transition. In some embodiments, solidification occurs without requiring mixing the liquid embolic composition with another solution. In some, embodiments, solidification occurs in the absence of a chemical catalyst (compound, molecule, etc.)

The following are examples of performance characteristics of a successful liquid embolic composition and injection: 1) easy (e.g., manual) injection through a delivery device such as a microcatheter or needle, (due to low viscosity of emulsion), 2) the embolic composition precipitates upon contact with precipitation medium (e.g., an ionic solution) forming a solid cast, 3) the cast remains in a single piece, 4) there is no visible material (e.g., polymer debris) in the supernatant, and 5) the delivery device (e.g., the distal end of the microcatheter) easily separates from the solidified cast. In some embodiments, the liquid embolic composition exhibits at least two or more of the above-mentioned characteristics. In some embodiments, the liquid embolic composition exhibits at least three or more of the above-mentioned characteristics. In some embodiments, the liquid embolic composition exhibits at least four or more of the above-mentioned characteristics. In some embodiments, the liquid embolic composition exhibits all five of the above-mentioned characteristics. Evaluation of a liquid embolic composition may be performed, for instance, by injecting it into an ionic solution and observing its performance. The ionic solution has an ionic strength comparable to a physiological fluid. Examples of ionic solutions include but are not limited to a physiological fluid (e.g., blood), saline (e.g., phosphate-buffered saline.) In some embodiments, the ionic solution is a physiological fluid. In some embodiments, the ionic solution is blood.

In some embodiments, the liquid embolic composition is free of an organic solvent (e.g., DMSO). An organic solvent-free composition refers to the absence of an organic solvent used to solubilize the polymers. Compositions may be considered solvent-free even if they have residual solvent (e.g., from manufacturing or processing steps, or from solubilizing an additive). In some embodiments, the solvent-free compositions described herein have an amount of residual organic solvent of less than 1 wt %, less than 0.5 wt %, less than 0.25 wt %, less than 0.1 wt %, or less than 0.05 wt %.

In some embodiments, the liquid embolic composition for occluding a treatment site comprises a biocompatible polymer, and water; wherein the biocompatible polymer comprises a hydrophobic polymer and a charged head group, wherein the hydrophobic polymer is poly(vinyl acetate), poly(styrene), or poly(methyl methacrylate), and the charged head group is derived from a radical initiator selected from the group consisting of 2,2′-azobis(2-methylpropionamidine) dihydrochloride, 2,2′-azobis[2-(2-imidazolin-2-yl)propane]dihydrochloride, potassium persulfate, ammonium persulfate, and sodium persulfate, and wherein the liquid embolic composition is an emulsion comprising particles of the biocompatible polymer suspended in the water, and the liquid embolic composition forms a solid when in contact with an ionic solution (such as blood).

In some embodiments, the liquid embolic composition for occluding a treatment site comprises a biocompatible polymer, and water; wherein the biocompatible polymer comprises a hydrophobic polymer and a charged head group, wherein the hydrophobic polymer is optionally substituted poly(vinyl acetate), and the charged head group is derived from a radical initiator selected from the group consisting of 2,2′-azobis(2-methylpropionamidine)dihydrochloride, and 2,2′-azobis[2-(2-imidazolin-2-yl)propane]dihydrochloride, and wherein the liquid embolic composition is an emulsion comprising particles of the biocompatible polymer suspended in the water, and the liquid embolic composition forms a solid when in contact with an ionic solution (such as blood).

In some embodiments, the biocompatible polymer is a polymerization product of a mixture comprising: a polar radical initiator, water, and hydrophobic monomers selected from the group consisting of acrylate, vinyl acetate, styrene, acrylonitrile, and vinyl chloride, each of which is optionally substituted, wherein the mixture comprises a molar ratio of polar radical initiators to hydrophobic monomers (initiator: monomer) selected from the group consisting of 1:1000 to 1:5000, 1:2000 to 1:5000, and 1:2000 to 1:4000; and the viscosity of the liquid emulsion at 25° C. is 1.0 cP to 3 cP.

In some embodiments, the biocompatible polymer is a polymerization product of a mixture comprising: a charged radical initiator, water, and hydrophobic monomers selected from the group consisting of acrylate, vinyl acetate, styrene, acrylonitrile, and vinyl chloride, each of which is optionally substituted, wherein the mixture comprises a molar ratio of polar radical initiators to hydrophobic monomers (initiator: monomer) is about 1:2500 to about 1:3500; and the viscosity of the liquid emulsion at 25° C. is 1.0 cP to 3 cP.

In some embodiments, the biocompatible polymer is a polymerization product of a mixture comprising: a charged radical initiator, water, and hydrophobic monomers selected from the group consisting of acrylate and vinyl acetate, each of which is optionally substituted, wherein the mixture comprises a molar ratio of polar radical initiators to hydrophobic monomers (initiator: monomer) is about 1:2500 to about 1:3500; and the viscosity of the liquid emulsion at 25° C. is 1.0 cP to 3 cP.

### II. TREATMENT SYSTEMS

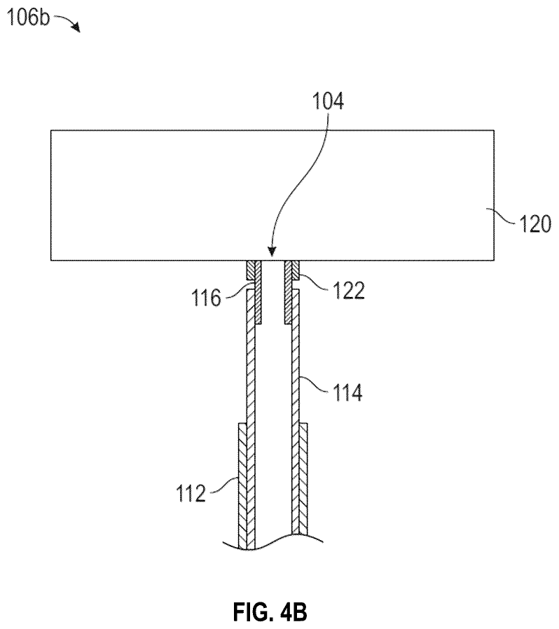

FIG. 4A shows a treatment system 100 (“system 100”) configured in accordance with embodiments of the present technology. The system 100 can be used in the treatment of many different types of diseases and conditions, such as arteriovenous malformations (e.g., brain arteriovenous malformations), arteriovenous fistulas, tumors (e.g., via occlusion of vessel(s) feeding a tumor), chronic subdural hematomas (e.g., via occlusion of the middle meningeal artery), perivascular leaks, varicose veins (e.g., via occlusion of one or more truncal veins such as the great saphenous vein), hemorrhoids, and sealing endoleaks adjacent to artificial heart valves, covered stents, and abdominal aortic aneurysm devices.

As shown in FIG. 4A, the system 100 includes a delivery system 102 including a distal outlet 104 for delivering an embolic composition to a treatment site, and an embolic kit 150 for preparing the embolic composition 152. The embolic composition 152 can be any material suitable for forming a solid or semi-solid viscoelastic structure (e.g., a cast) that partially or completely occludes the treatment site, as discussed further below.

The delivery system 102 has a proximal portion 106a configured to be extracorporeally positioned during treatment and a distal portion 106b including the distal outlet 104 that is configured to be intravascularly positioned at or within a treatment site (e.g., a vascular defect or other lesion). The delivery system 102 may include a handle 108 at the proximal portion 106a and a plurality of elongate shafts extending between the handle 108 and the distal portion 106b. In some embodiments, for example as shown in FIG. 4A, the delivery system 102 may include a first elongate shaft 110 (such as a guide catheter or balloon guide catheter), a second elongate shaft 112 (such as a microcatheter) configured to be slidably disposed within a lumen of the first elongate shaft 110, and a third elongate shaft 114 configured to be slidably disposed within a lumen of the second elongate shaft 112. In some embodiments, the delivery system 102 does not include the first elongate shaft 110.

The second elongate shaft 112 can be constructed to track over a conventional guidewire and into vasculature to access the treatment site. The second elongate shaft 112 may also be chosen according to several standard designs that are generally available. For example, the second elongate shaft 112 can have a length that is at least 125 cm long, and more particularly may be between about 125 cm and about 175 cm long. The second elongate shaft 112 can have an inner diameter less than or equal to 0.006 inches (0.015 cm), 0.011 inches (0.028 cm), 0.015 inches (0.038 cm), 0.017 inches (0.043 cm), 0.021 inches (0.053 cm), or 0.027 inches (0.069 cm).

The third elongate shaft 114 can be movable within the first and/or second elongate shafts 110, 112 to position the distal outlet 104 at a desired location. The third elongate shaft 114 can be sufficiently flexible to enable manipulation (e.g., advancement and/or retraction) through tortuous passages. Tortuous passages can include, for example, catheter lumens, microcatheter lumens, blood vessels, urinary tracts, biliary tracts, and airways. The third elongate shaft 114 can be formed of any material and in any dimensions suitable for the task(s) for which the system 100 is to be employed. In some embodiments, at least the distal portion of the third elongate shaft 114 can comprise a flexible metal hypotube. The hypotube, for example, can be laser cut along all or a portion of its length to impart increased flexibility. In some embodiments, the third elongate shaft 114 can be surrounded over some or all of its length by a lubricious coating, such as polytetrafluoroethylene (PTFE). The third elongate shaft 114 can have an inner diameter less than or equal to 0.006 inches (0.015 cm), 0.011 inches (0.028 cm), 0.015 inches (0.038 cm), 0.017 inches (0.043 cm), 0.021 inches (0.053 cm), or 0.027 inches (0.069 cm).

Although FIG. 4A illustrates the delivery system 102 with three elongate shafts 110, 112, and 114, in other embodiments, some of the elongate shafts may be omitted or the delivery system 102 may include additional elongate shafts.

The embolic kit 150 includes an embolic composition 152 (e.g., a liquid embolic composition as described in Section I above) and an injector 154 configured to be fluidly coupled to a proximal portion of the delivery system 102 for injection of the embolic composition 152 into a treatment site. For example, a distal end portion of the injector 154 can be fluidly coupled to a proximal end portion of the third elongate shaft 114 via a port 118. The port 118 can be located at the proximal portion 106a of the delivery system 102, such as on or proximal to the handle 108.

Referring again to FIG. 4A, the injector 154 can be configured to pressurize the embolic composition 152 to a pressure that is sufficiently high to push the embolic composition 152 through the components of the delivery system 102 (e.g., through the lumen of the third elongate shaft 114) and into the treatment site to fill and occlude the treatment site. As described further in Section I above, the embolic composition 152 is liquid so that relatively low pressures are needed to inject the embolic composition 152 through the delivery system 102, e.g., the embolic composition 152 can be injectable by hand through a standard disposable syringe. In some embodiments, the maximum pressure needed to inject the embolic composition 152 is less than or equal to 5000 psi, 4000 psi, 3000 psi, 2000 psi, 1000 psi, 500 psi, 200 psi, or 100 psi.

Referring next to FIG. 4B, the system 100 can optionally include an occlusive device 120 (also referred to herein as an “occlusive member,” “neck cover,” or a “neck protection device”-shown schematically) at the distal portion 106b. The occlusive device 120 may be used, for example, to prevent the embolic composition 152 from leaking out of the treatment site. For example, in embodiments where the system 100 is used to treat an aneurysm, the embolic composition 152 can be delivered to a space between the occlusive device 120 and the dome of the aneurysm to fill and occlude the aneurysm cavity. The occlusive device 120 prevents migration of the embolic composition 152 into the parent vessel, and together the occlusive device 120 and embolic composition 152 prevent blood from flowing into the aneurysm. In some embodiments, bioabsorption of the embolic composition 152 (in embodiments where the embolic composition 152 is biodegradable) and/or endothelialization of the occlusive device 120 may cause the aneurysm wall to fully degrade, leaving behind a successfully remodeled (aneurysm free) region of the blood vessel.

In some embodiments, the occlusive device 120 is configured to be detachably coupled to the delivery system 102, and the delivery system 102 is configured to intravascularly position the occlusive device 120 within the treatment site. Representative examples of occlusive devices suitable for use with the system 100 are described in U.S. Pat. Nos. 8,142,456, 9,855,051, 10,327,781, U.S. Patent Application Publication No. 2020/0187953, U.S. Patent Application Publication No. 2021/0128169, and U.S. Patent Application Publication No. 2021/0153872, the disclosures of which are incorporated by reference herein in their entirety.

The occlusive device 120 may comprise an expandable element having a low-profile or constrained state while positioned within a catheter (such as the second elongate shaft 112) for delivery to the treatment site and an expanded, deployed state for positioning within the treatment site. In some embodiments, the occlusive device 120 includes a mesh and a connector 122 coupled to the mesh. The connector 122 is configured to be coupled to one or more components of the delivery system 102, such as the third elongate shaft 114 and/or an extension 116. The mesh can be formed of a resilient material and shape set such that upon exiting the second elongate shaft 112, the mesh self-expands to a predetermined shape. The mesh can have any shape or size in the expanded state that enables the mesh to cover the treatment site (e.g., an aneurysm neck). The mesh can have a porosity sufficient to prevent leakage of the embolic composition 152 out of the treatment site (e.g., a parent vessel of an aneurysm).

In some embodiments, the mesh is formed of a plurality of braided filaments that have been heat-set to assume a predetermined shape when released from the constraints of the delivery catheter. The mesh may be formed of metal wires, polymer wires, or both, and the wires may have shape memory and/or superelastic properties. The mesh may be formed of 24, 32, 36, 48, 64, 72, 96, 128, or 144 filaments. The mesh may be formed of a range of filament or wire sizes, such as wires having a diameter of from about 0.0004 inches to about 0.0020 inches, or of from about 0.0009 inches to about 0.0012 inches. In some embodiments, each of the wires or filaments have a diameter of about 0.0004 inches, about 0.0005 inches, about 0.0006 inches, about 0.0007 inches, about 0.0008 inches, about 0.0009 inches, about 0.001 inches, about 0.0011 inches, about 0.0012 inches, about 0.0013 inches, about 0.0014 inches, about 0.0015 inches, about 0.0016 inches, about 0.0017 inches, about 0.0018 inches, about 0.0019 inches, or about 0.0020 inches. In some embodiments, all of the filaments of the braided mesh may have the same diameter. For example, in some embodiments, all of the filaments have a diameter of no more than 0.001 inches. In some embodiments, some of the filaments may have different cross-sectional diameters. For example, some of the filaments may have a slightly thicker diameter to impart additional strength to the braid. In some embodiments, some of the filaments can have a diameter of no more than 0.001 inches, and some of the filaments can have a diameter of greater than 0.001 inches. The thicker filaments may impart greater strength to the braid without significantly increasing the device delivery profile, with the thinner wires offering some strength while filling out the braid matrix density. In some embodiments, the mesh can be a non-braided structure, such as a laser-cut stent.

In other embodiments, however, the occlusive device 120 may be replaced with a different type of device (e.g., a flow diverter) or the embolic composition 152 can be used without any occlusive device (e.g., if physiological fluid flow at the treatment site is expected to be sufficiently low such that leakage of the embolic composition 152 is not a significant concern).

### III. EXAMPLES

The following examples are included to further describe some aspects of the present technology and should not be used to limit the scope of the technology.

### A. Example 1: Preparation of the Polymer Emulsion

This example describes the preparation of 11 polymer emulsion compositions comprising either vinyl acetate, styrene, or methyl methacrylate, with varying concentrations of monomer and initiator.

### 1. Materials

The material used to prepare exemplary compositions include: 1) deionized water (90%), 2) the positively charged radical initiator 2,2′-Azobis(2-methylpropionamidine) dihydrochloride, and 3) vinyl monomers, including vinyl acetate, styrene, and methyl methacrylate. The amounts of water, initiator and monomers are summarized below in Table 1, which can be found under section III.A.B.

### 2. Methods

Three stock solutions of 2,2′-Azobis(2-methylpropionamidine)dihydrochloride initiator in deionized water were prepared: 20 ml water and 0.2 g of initiator (compositions #2-4); 50 ml water and 0.09 g of initiator (compositions #5-6); and 40 ml water and 0.088 g of initiator (compositions #7-11).

The water, initiator and monomers were combined in a round bottom flask in the amounts summarized in Table 1. Nitrogen gas was bubbled into the mixture for 20 minutes to remove oxygen. Then, the initiator was activated by heating the flask to a temperature of 65-70° C. This temperature was maintained for at least about 5 hours to overnight. The mixture formed a polymer emulsion and was observed to have a milky consistency. See FIG. 2. The polymerization reaction was terminated by cooling the mixture to room temperature and exposing it to air.

In some embodiments, unreacted monomer units are removed by dialysis. In some embodiments, a concentrated emulsion is obtained by centrifugation.

### B. Example 2: Evaluation of the Polymer Emulsion Performance

This example describes pipetting the polymer emulsions prepared in Example 1 into an ionic solution and observed behavior of the mixture.

The polymer emulsions were tested by injecting the emulsion in a high ionic content liquid (phosphate-buffered saline solution or 0.9% saline). Exposing the suspended polymer particles to the ionic solution caused the polymer to aggregate resulting in the polymer precipitating and forming a solid mass. The ease of injecting the polymer was assessed. The cohesiveness of the precipitate was assessed by probing the solid mass by manipulating it with a spatula.

Subjecting the solid mass (cast) to compression tests using DMA (Dynamic mechanical Analysis instrument) or shear tests in a rheometer provides information on the cohesive character of the material. Utilizing DMA, the elastic properties of the cast are measured (e.g., elastic modulus under compression). Elastic and storage modulus as well as yield stress of the cast is measured with a rheometer.

Table 1 summarizes the amounts of water, monomer, and initiator for each attempted polymerization example, and the observed behavior of the emulsions when delivered to an ionic solution. The viscosity of emulsions 1-4 were 1.11 cP to 1.21 cP as measured using a Brookfield viscometer at 25° C. (Composition #1, 1.11 cP; Composition #2, 1.26 cP; Composition #3, 1.31 cP; Composition #4, 1.28 cP, Composition #5, 1.21).

TABLE 1

| | | | | | | Observations Post- |
|---|---|---|---|---|---|---|
| | Water | Monomer | Initiator | Initiator:Monomer | | Injection into Saline or |
| # | (mL) | (mL) | (g) | Ratio | Polymerization Outcome | PBS |
| 1 | 90 | 10 | 0.03 | 1:1050 | Milky suspension | Precipitates in PBS. |
| | | vinyl acetate | (1 mL stock solution) | | | Forms cloudy solution in |
| | | | | | | saline. |
| 2 | 29 | 3 | 0.01 | 1:950 | Milky suspension | Precipitation took place, |
| | | vinyl acetate | (1 mL stock solution) | | | cohesive weak cast |
| 3 | 24 | 3 | 0.06 | 1:160 | Milky suspension | Precipitation took place, |
| | | vinyl acetate | (6 mL stock solution) | | | cohesive weak cast |
| 4 | 30 | 3 | 0.003 | 1:3150 | Took a few minutes to turn | Precipitation took place, |
| | | vinyl acetate | (0.3 mL stock solution) | | milky | cohesive cast. Precipitated |
| | | | | | | in both saline and PBS. |
| 5 | 27 | 3 | 0.001 | 1:9450 | Took several minutes to | Non-cohesive cast |
| | | vinyl acetate | (0.56 mL stock solution) | | turn milky | |
| 6 | 27 | 3 | 0.0003 | 1:31500 | No polymerization | N/A |
| | | vinyl acetate | (0.17 mL stock solution) | | | |
| 7 | 20 | 2.2 | 0.0066 | 1:870 | Turned milky within | Precipitated in saline, not |
| | | styrene | (3 mL stock solution) | | minutes | cohesive |
| 8 | 20 | 2.2 | 0.0022 | 1:2600 | No polymerization | N/A |
| | | styrene | (1 mL stock solution) | | | |
| 9 | 20 | 2.2 | 0.0066 | 1:900 | Milky suspension | Precipitated in saline, not |
| | | methyl | (3 mL stock solution) | | with some | cohesive |
| | | methacrylate | | | solid precipitate after the | |
| | | | | | end of the reaction | |
| 10 | 20 | 2.2 | 0.0022 | 1:2700 | White emulsion was formed | Precipitated in saline, not |
| | | methyl | (1 mL stock solution) | | within minutes | cohesive. |
| | | methacrylate | | | | |
| 11 | 20 | 2.2 | 0.00088 | 1:6800 | No polymerization | N/A |
| | | methyl | (0.4 mL stock solution) | | | |
| | | methacrylate | | | | |

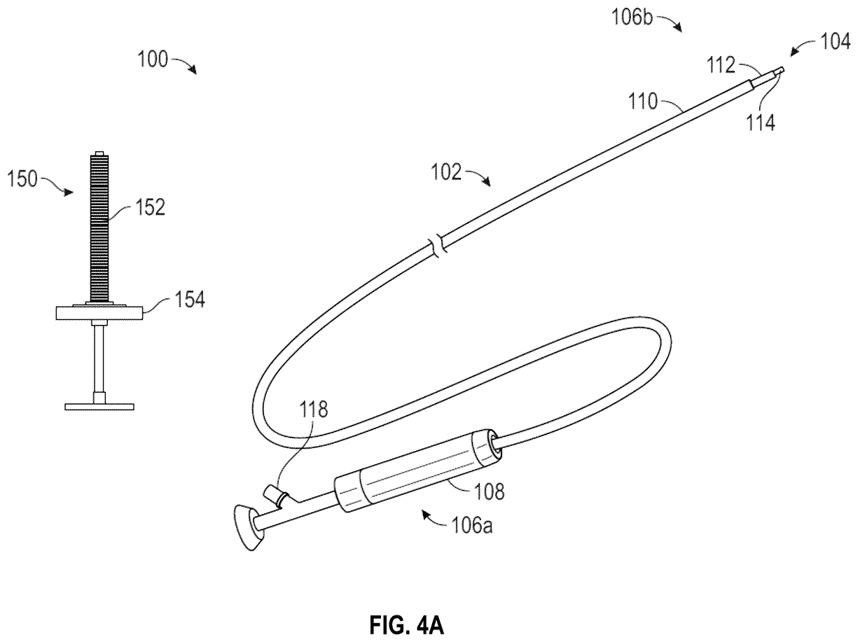

FIG. 3A shows a photo demonstrating the injection of the composition example #4 into 0.9% saline solution at room temperature. As the liquid polymer emulsion comes in contact with the saline solution, it precipitates immediately to form a solid cast. The cast remains substantially in a single piece after probing the solid and moving it around with a spatula, FIG. 3B, indicating indicates a cohesive nature.

The initiator to monomer ratio impacted the behavior of the emulsion. A high initiator: monomer ratio resulted in an emulsion that did not precipitate well in saline/PBS. Lower molecular weight chains may have led to a smaller particle size (polymer aggregates) that remained suspended as part of the emulsion and did not precipitate in an ionic solution to form a solid cast.

On the other hand, if the initiator: monomer ratio is too low, either polymerization does not take place, as exemplified by compositions 6 (vinyl acetate), 8 (styrene), and 11 (methyl methacrylate). Alternatively, the resulting long polymer chains may be too hydrophobic to remain suspended, resulting in precipitation during polymerization.

For example, with respect to the vinyl acetate polymerizations where initiator to monomer molar ratios were higher than about 1:1000 (e.g., samples 1, 2, and 3), non-cohesive casts were formed due to likely low molecular weight of the chain and thus, particle size. When initiator to monomer ratios were less than about 1:10000 (e.g., samples 5 and 6), poor precipitation in ionic solution took place. This result is potentially due to low polymerization yield (not many active radicals).

Other variations of the emulsion composition were not as successful. For example, copolymerizing the hydrophobic first monomer (e.g., styrene or MMA) with a second hydrophilic monomer (e.g., hydroxyethyl acrylate, acrylic acid, and NIPAM) resulted in making the emulsion too hydrophilic. These variations led to poor precipitation behavior in ionic solutions.

With respect to monomer concentration, a concentration of about 10% v/v of monomer to water was used.

### IV. MISCELLANEOUS

The present disclosure is applicable to many applications and/or approaches, such as treatment of arteriovenous malformations (e.g., brain arteriovenous malformations), arteriovenous fistulas, tumors (e.g., via occlusion of vessel(s) feeding a tumor), and chronic subdural hematomas (e.g., via occlusion of the middle meningeal artery) among other examples. Additionally, several other embodiments of the disclosure can have different configurations, components, or procedures than those described herein. A person of ordinary skill in the art, therefore, will accordingly understand that the disclosure can have other embodiments with additional elements, or the disclosure can have other embodiments without several of the features shown and described above with reference to FIGS. 1A-4B. The descriptions of embodiments disclosed herein are not intended to be exhaustive or to limit the disclosure to the precise form disclosed above.

Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

It will also be appreciated that specific embodiments have been described herein for purposes of illustration, but that various modifications may be made without deviating from the disclosure. Further, while advantages associated with certain embodiments disclosed herein have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

Where the context permits, singular or plural terms may also include the plural or singular term, respectively. As used herein, the terms “generally,” “substantially,” “about,” and similar terms are used as terms of approximation and not as terms of degree, and are intended to account for the inherent variations in measured or calculated values that would be recognized by those of ordinary skill in the art. As used herein, the term “about” is used synonymously with the term “approximately.” Illustratively, the use of the term “about” with regard to an amount indicates values slightly outside the cited values, e.g., plus or minus 10%, plus or minus 5%, plus or minus 2%, plus or minus 1%, plus or minus 0.5%, plus or minus 0.2%, or plus or minus 0.1%.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list.

Additionally, the term “comprising” is used throughout to mean including at least the recited feature(s) such that any greater number of the same feature and/or additional types of other features are not precluded.

### V. Clauses

The present technology is illustrated, for example, according to various aspects described below. Various examples of aspects of the present technology are described as numbered clauses (1, 2, 3, etc.) for convenience. These various aspects are provided as examples and do not limit the subject technology.

Clause I-1: A liquid embolic composition for occluding a treatment site comprising: a biocompatible polymer, and water; wherein the biocompatible polymer comprises a hydrophobic polymer and a polar head group, and wherein the liquid embolic composition is an emulsion comprising particles of the biocompatible polymer suspended in the water, and wherein the liquid embolic composition forms a solid when in contact with an ionic solution.

Clause I-2: The liquid embolic composition of Clause I-1, wherein the hydrophobic polymer is selected from the group consisting of polyacrylate, polyvinyl acetate, polystyrene, polyacrylonitrile, and polyvinyl chloride, each of which is optionally substituted.

Clause I-3: The liquid embolic composition of Clause I-2, wherein the polyacrylate is selected from the group consisting of poly(methyl methacrylate), poly(butyl methacrylate), poly(hexyl methacrylate), poly(octyl methacrylate), poly(2-ethylhexyl methacrylate), poly(benzyl methacrylate), poly(cyclohexyl methacrylate), poly(hexadecyl methacrylate), poly(octadecyl acrylate), and poly(phenyl methacrylate), each of which is optionally substituted.

Clause I-4: The liquid embolic composition of Clause I-2, wherein the polystyrene is substituted with one or more substituents selected from the group selected methyl, halogen, hydroxy, acetoxy, methyl, butyl, ethoxy, and fluoro.

Clause I-5: The liquid embolic composition of any one of Clauses I-1 to I-4, wherein the polar head group is derived from a radical initiator.

Clause I-6: The liquid embolic composition of Clause I-5 wherein the radical initiator is selected from the group consisting of: 2,2′-azobis(2-methylpropionamidine) dihydrochloride, 2,2′-azobis[2-(2-imidazolin-2-yl)propane]dihydrochloride, potassium persulfate, ammonium persulfate, sodium persulfate, 2,2′-azobis[2-methyl-n-(2-hydroxyethyl)propionamide], 4,4′-azobis(4-cyanovaleric acid), 2,2′-azobis[n-(2-carboxyethyl)-2-methylpropionamidine]tetrahydrate, and 2,2′-azobis[2-(2-imidazolin-2-yl)propane].

Clause I-7: The liquid embolic composition of any one of Clauses I-1 to I-6, wherein the water is at least 80% deionized water.

Clause I-8: The liquid embolic composition of any one of Clauses I-1 to I-7, wherein the ionic solution is saline or a physiological fluid.

Clause I-9: The liquid embolic composition of Clause I-8, wherein the physiological fluid is blood.

Clause I-10: The liquid embolic composition of any one of Clauses I-1 to I-9, wherein the biocompatible polymer is a polymerization product of a mixture comprising: a polar radical initiator, water, and hydrophobic monomers selected from the group consisting of acrylate, vinyl acetate, styrene, acrylonitrile, and vinyl chloride, each of which is optionally substituted.

Clause I-11: The liquid embolic composition of Clause I-10, wherein the mixture comprises a molar ratio of polar radical initiators to hydrophobic monomers (initiator: monomer) selected from the group consisting of 1:100 to 1:30000, 1:1000 to 1:20000, 1:1000 to 1:10000, 1:1000 to 1:5000, 1:2000 to 1:20000, 1:2000 to 1:10000, 1:2000 to 1:5000, 1:3000 to 1:20000, 1:3000 to 1:10000, 1:3000 to 1:5000, and 1:2000 to 1:4000.

Clause I-12: The liquid embolic composition of any one of Clauses I-10 to I-11, wherein the mixture comprises hydrophobic monomers in a concentration of 10% v/v.

Clause I-13: The liquid embolic composition of any one of Clauses I-1 to I-12, wherein the composition has a viscosity at 25° C. of 1 cP to 10 cP.

Clause I-14: The liquid embolic composition of any one of Clauses I-1 to I-12, wherein the viscosity of the liquid emulsion at 25° C. is 1.0 cP to 3 cP.

Clause I-15: The liquid embolic composition of any one of Clauses I-1 to I-14, further comprising a non-ionic contrast agent.

Clause I-16: The liquid embolic composition of Clause I-15, wherein the non-ionic contrast agent is selected from the group consisting of iopamidol, iohexol, iopromide, ioversol, ioxilan, iodixanol, and lipiodol.

Clause I-17: The liquid embolic composition of any one of Clauses I-1 to -16, for use in a neurovascular or peripheral embolization procedure.

Clause I-18: The liquid embolic composition of Clause I-17, wherein the neurovascular or peripheral embolization procedure is selected from the group consisting of middle meningeal artery embolization, arteriovenous malformation embolization, arteriovenous fistula embolization, and tumor embolization.

Clause I-19: A solid embolic composition formed by contacting the liquid embolic composition of any one of Clauses I-1 to I-18 with an ionic solution, wherein the liquid embolic composition forms a soft solid.

Clause I-20: The solid embolic composition of Clause I-19, wherein the soft solid remains cohesive in the ionic solution.

Clause I-21: The solid embolic composition of Clause I-19 or I-20, wherein the ionic solution is saline or a physiological fluid.

Clause I-22: A method for occluding a treatment site, the method comprising: delivering the liquid embolic composition of any one of Clauses I-1 to I-18 into or near the treatment site to occlude the treatment site.

Clause I-23: The method of Clause I-22, wherein the liquid embolic composition is delivered into the treatment site via injection through a catheter.

Clause I-24: The method of Clause I-22 or I-23, wherein the treatment site is selected from the group consisting of a middle meningeal artery, an arteriovenous malformation, an arteriovenous fistula, and a tumor.

I/We claim:

1. A liquid embolic composition for occluding a treatment site comprising:
a biocompatible polymer, and
water;
wherein the biocompatible polymer comprises a hydrophobic polymer and a polar head group, and
wherein the liquid embolic composition is an emulsion comprising particles of the biocompatible polymer suspended in the water, and
wherein the liquid embolic composition forms a solid when in contact with an ionic solution.

2. The liquid embolic composition of claim 1, wherein the hydrophobic polymer is selected from the group consisting of polyacrylate, polyvinyl acetate, polystyrene, polyacrylonitrile, and polyvinyl chloride, each of which is optionally substituted.

3. The liquid embolic composition of claim 2, wherein the polyacrylate is selected from the group consisting of poly(methyl methacrylate), poly(butyl methacrylate), poly(hexyl methacrylate), poly(octyl methacrylate), poly(2-ethylhexyl methacrylate), poly(benzyl methacrylate), poly(cyclohexyl methacrylate), poly(hexadecyl methacrylate), poly(octadecyl acrylate), and poly(phenyl methacrylate), each of which is optionally substituted.

4. The liquid embolic composition of claim 2, wherein the polystyrene is substituted with one or more substituents selected from the group selected methyl, halogen, hydroxy, acetoxy, methyl, butyl, ethoxy, and fluoro.

5. The liquid embolic composition of claim 1, wherein the polar head group is derived from a radical initiator.

6. The liquid embolic composition of claim 5 wherein the radical initiator is selected from the group consisting of:
2,2′-azobis(2-methylpropionamidine)dihydrochloride,
2,2′-azobis[2-(2-imidazolin-2-yl)propane]dihydrochloride,
potassium persulfate,
ammonium persulfate,
sodium persulfate,
2,2′-azobis[2-methyl-n-(2-hydroxyethyl)propionamide],
4,4′-azobis(4-cyanovaleric acid),
2,2′-azobis[n-(2-carboxyethyl)-2-methylpropionamidine]tetrahydrate, and
2,2′-azobis[2-(2-imidazolin-2-yl)propane].

7. The liquid embolic composition of claim 1, wherein the ionic solution is saline or a physiological fluid.

8. The liquid embolic composition of claim 7, wherein the physiological fluid is blood.

9. The liquid embolic composition of claim 1, wherein the biocompatible polymer is a polymerization product of a mixture comprising:
a polar radical initiator,
water, and
hydrophobic monomers selected from the group consisting of acrylate, vinyl acetate, styrene, acrylonitrile, and vinyl chloride, each of which is optionally substituted.

10. The liquid embolic composition of claim 9, wherein the mixture comprises hydrophobic monomers in a concentration of 10% v/v.

11. The liquid embolic composition of claim 1, wherein the liquid embolic composition has a viscosity at 25° C. of 1 cP to 10 cP.

12. The liquid embolic composition of claim 1, further comprising a non-ionic contrast agent.

13. The liquid embolic composition of claim 1, for use in a neurovascular or peripheral embolization procedure.

14. The liquid embolic composition of claim 13, wherein the neurovascular or peripheral embolization procedure is selected from the group consisting of middle meningeal artery embolization, arteriovenous malformation embolization, arteriovenous fistula embolization, and tumor embolization.

15. A solid embolic composition formed by contacting the liquid embolic composition of claim 1 with an ionic solution, wherein the liquid embolic composition forms a soft solid.

16. The solid embolic composition of claim 15, wherein the soft solid remains cohesive in the ionic solution.

17. The solid embolic composition of claim 15, wherein the ionic solution is saline or a physiological fluid.

18. A method for occluding a treatment site, the method comprising:
delivering the liquid embolic composition of claim 1 into or near the treatment site to occlude the treatment site.

19. The method of claim 18, wherein the liquid embolic composition is delivered into the treatment site via injection through a catheter.

20. The method of claim 18, wherein the treatment site is selected from the group consisting of a middle meningeal artery, an arteriovenous malformation, an arteriovenous fistula, and a tumor.
